(19) United States
(12) Patent Application Publication
(10) Pub. No.: US 2023/0133259 A1
(43) Pub. Date: May 4, 2023

(54) INJURY PROTECTION DEVICE FOR INTRAVENOUS LINES

(71) Applicant: LANKENAU INSTITUTE FOR MEDICAL RESEARCH, Wynnewood, PA (US)

(72) Inventor: Boris Aronzon, Gouldsboro, PA (US)

(21) Appl. No.: 17/977,384

(22) Filed: Oct. 31, 2022

Related U.S. Application Data
(60) Provisional application No. 63/274,730, filed on Nov. 2, 2021.

(51) Int. Cl. A61M 25/02

(52) U.S. Cl. CPC A61M 25/02; A61M 2025/0266; A61F 2013/00412

(57) ABSTRACT

The present invention provides a protective device that protects the patient's skin and underlying tissue from pressure and injury resulting from pressure on the skin/tissue and/or compression of nerves by IV lines associated with medical procedures, medication administration, for nutritional purposes, and the like. In one embodiment, a protective device in accordance with the present invention comprises a body defining a window dimensioned to receive a portion of an IV line, said body having a lower surface having an area greater than said portion of said IV line. The window may be internal to, or open to, a peripheral edge of the body. Adhesive may be applied to the upper and/or lower surface of the body. A securing tab or catch may be provided to secure the IV line to the body.

### CROSS-REFERENCE TO RELATED APPLICATION

This application claims the benefit of priority of U.S. Provisional Patent Application No. 63/274,730, filed Nov. 2, 2021, the entire disclosure of which is hereby incorporated herein by reference.

### FIELD OF THE INVENTION

The present invention relates generally to devices for use in the operating theater or other vise in connection with medical treatment or care in which the patient requires intravenous (IV) placement for a prolonged period of time or has fragile skin that may deteriorate from being in contact with IV tubing or ports, and more particularly, to a protective device for use to protect the arm, hand and/or other limb/body regions of a patient from skin/tissue and/or nerve injuries caused by contact of medical procedure-related intravenous (IV) lines with the patient's skin, which creates pressure points during medical treatment that can lead to ulcers and other injuries.

### DISCUSSION OF RELATED ART

Various surgeries, medical treatments, and other aspects of medical and health care (collectively referred to herein as “procedures”) involve the use of intravenous (IV) lines, which generally have an associated needle, tubing, tubing hubs, ports, Luer locks, Y-piece connections and/or other fittings. Often, as part of proper medical procedure and/or convenience, these IV lines are positioned to extend along, rest upon and/or abut the patient's hand and/or arm regions.

While discrete medical procedures may be relatively short in duration, e.g., 3-6 hours, in other health care settings, such as in hospital, hospice, rehabilitation and/or nursing home-type care settings, IVs may be placed due to chronic conditions for much longer periods of time, such as a period of days or more. Such prolonged IV use increases the likelihood that skin/pressure ulcers and/or nerve or other injuries (e.g., contact dermatitis, infection, laceration, itching) will occur from pressure on the skin by the associated IV line components.

Skin injuries/wounds/sores are undesirable, and tend to be expensive for the care facility. Skin injuries may be particularly likely to occur in elderly fragile patients or patients with peripheral vascular disease, neuropathy, obesity, diabetes, smoking history or advanced age, where skin perfusion can be compromised. Additionally, nerve injuries are undesirable for many reasons, including neuropathy and/or other impacts upon the patient, the possible need for corrective surgery and/or procedures to aid in recovery from the injury, or extensive and costly rehabilitation treatment, dissatisfaction of the patient, and potential medical malpractice or similar claims, all of which are undesirable.

Accordingly, there is a need for a device that is compatible with conventional IV lines and associated care/procedures that protects the patient's skin and other tissues from sores, nerve compression and/or injury resulting from pressure on the skin by IV lines associated with such procedures.

### SUMMARY

The present invention provides a protective device that defends/protects the patient's skin and underlying tissue from pressure and injury resulting from pressure on the skin/tissue and/or compression of nerves by IV lines associated with medical procedures, medication administration, for nutritional purposes, and the like. In one embodiment, a protective device in accordance with the present invention comprises a body defining a window dimensioned to receive a portion of an IV line, said body having a lower surface having an area greater than said portion of said IV line. The window may be internal to, or open to, a peripheral edge of the body. Adhesive may be applied to the upper and/or lower surface of the body. A securing tab or catch may be provided to secure the IV line to the body. The body may have a uniform thickness or may have a varying thickness, and be generally wedge-shaped.

### BRIEF DESCRIPTION OF THE FIGURES

An understanding of the following description will be facilitated by reference to the attached drawings, in which:

FIG. 1 is a perspective view of a skin and nerve injury protection device in accordance with an exemplary embodiment of the present invention, shown in use on a human hand;

FIG. 2 is a perspective view of the device of FIG. 1;

FIG. 3 is a perspective view of another exemplary device in accordance with an alternative embodiment of the present invention;

FIG. 4 is a perspective view of yet another exemplary device in accordance with an alternative embodiment of the present invention;

FIG. 5 is a perspective view of the device of FIG. 3 or FIG. 4, shown in use on a human hand;

FIG. 6 is a top view of yet another exemplary device in accordance with an alternative embodiment of the present invention; and

FIG. 7 is a top view of yet another exemplary device in accordance with an alternative embodiment of the present invention.

### DETAILED DESCRIPTION

The present invention provides an IV skin and nerve injury protection device (also referred to herein as a “protective device” or “device”) that is configured to protect the patient's hands, arms (or other limbs or body parts) from pressure exerted by IV lines on the skin and other tissues and resulting compression of the skin, and possibly, nerves, and from resulting skin sores, nerve compression and/or other injuries resulting from such pressure.

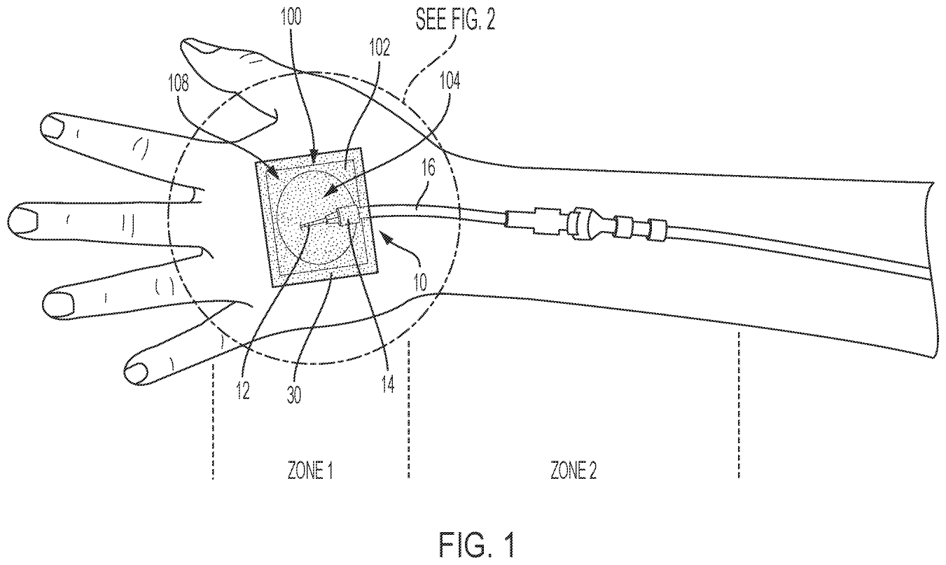

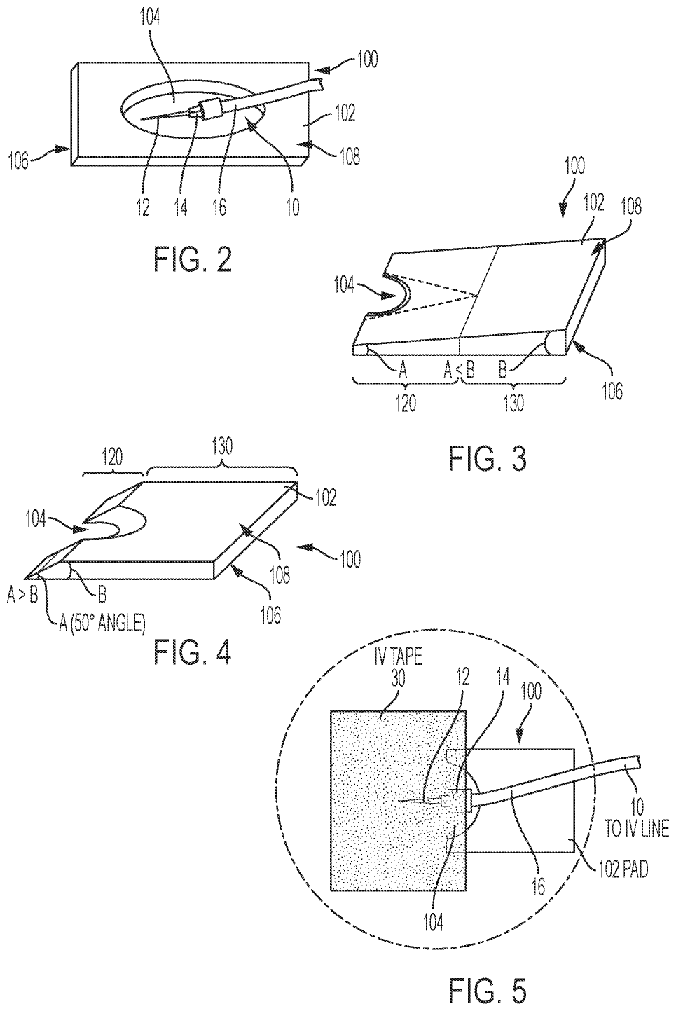

Referring now to FIGS. 1 and 2, an exemplary embodiment of an IV skin and nerve injury protection device 100 in accordance with the present invention is shown. As shown in FIGS. 1 and 2, the injury protection device 100 includes an elongated body 102. The body 102 may be formed as a unitary body defining a lower surface 106 having an area greater than the area of a hub/connector 14 of a conventional IV line. The lower surface 106 abuts the skin/bodily tissue during use, and the relatively larger area of the lower surface 106 allows pressure that would be exerted on the skin/bodily tissue by the hub/connector 14 to be spread over an area larger than the area of the hub/connector, to thereby reduce the pressure per unit area exerted on the skin/tissue, and thus to reduce the likelihood of resulting skin compression and skin and/or nerve injury, and thereby protect the patient against pressure ulcers/sores/nerve injuries, etc. Preferably, the area of the lower surface 106 is at least 1.5 times to at least 2.0 times greater than the area of the hub/connector 14. In certain embodiments, the overall device is approximately 9 to approximately 12 square inches in overall size.

The body 102 is preferably formed of a relatively flexible and readily compressible material, such as a foam material or gel, so as to readily bend and conform to any contours of a forearm, etc., and to act as a cushioning material. Further, the material, or at least a portion of the material, is preferably relatively thick, meaning at least 0.125 or 0.25 inches in thickness or having a thickness that is in the range of at least about 1 to about 5 times as thick as the thickness of the IV lines/connectors/fittings with which it is intended to be used, to facilitate the function of a portion of the material as cushioning, as described below. In other embodiments, the material may have any thickness suitable for decreasing pressure per square unit area to diminish the risk of the skin, nerve or tissue injury from unintended pressure from an IV line during prolonged procedures, etc. in which the IV line overlies bodily tissue, e.g., when it is compressed against the skin as a result of fixation to the skin or during patient positioning/limb fixation and/or tucking, e.g., for medical procedures.

Further, the elongated body may be constructed of a material that is readily cleanable, e.g., disinfectable and/or sterilizable, using cleaning methods of the type typically used for products used in the operating theater, and/or that resists absorption of bodily or other fluids, making it well-suited for use in the operating theater. By way of example, a closed-cell EPDM foam material may be used for this purpose. Alternatively, materials intended to be disposable, and not cleaned and reused, may be used.

In accordance with the present invention, the body 102 may define a window 104. The window defines a space for receiving a portion of the IV line, such as the catheter (or needle) and/or a hub/connector/fitting, as the body 102 is placed in close proximity to the point of entry of the IV line's needle, etc. into the skin. The body 102 then acts in somewhat of a wedge-like fashion, to be positioned between the IV line and the skin, to separate the IV line from the skin, to support the IV line above the skin, and to distribute pressure that would be exerted on the skin by the IV line (tubing, fittings, connectors or ports) over a relatively larger area.

In the exemplary embodiment of FIGS. 1 and 2, the window 104 is defined entirely internally to the body 102, so that material of the body 102 fully surrounds the window 104. As shown in the example of FIG. 2, the entire catheter 12, hub/connector 14, and a portion of the tubing 16 of the IV line 10 are positioned within the window 104 of the exemplary protective device 100. As shown in the example of FIG. 1, the entire catheter 12 (including the portion beneath the skin) is positioned within the window 104 of the exemplary protective device 100, and the hub/connector 14 is partially overlying and supported on an upper surface 108 of the body 102 of IV skin and nerve protective device 100. Additionally, a sheet of IV tape 30 (e.g., such as that commercially available from 3M under the name Tegaderm) is placed over the entire IV skin and nerve protective device 100 and a portion of the IV line 10. The placement of tape to fix/secure the IV line to the skin adjacent the penetration site is common practice, but it contributes to the exertion of pressure on the skin/tissue by the IV line and its components parts, such as the hub/connector 14. Here, undesirable pressure on the skin/tissue is mitigated by the skin and nerve protective device 100.

FIGS. 3 and 4 are perspective views exemplary alternative embodiments of an protective device 100 in accordance with the present invention. In these exemplary embodiments, the window 104 is not defined entirely internally to the body 102 (as in FIGS. 1 and 2), but rather the window 104 is open to a peripheral edge of the body 102, so that the window is formed somewhat like a notch in the peripheral edge and can be customized by the person placing the IV line, e.g., by using scissors to cut/trim the body 102. As with the embodiment of FIGS. 1 and 2, the window defines a space for receiving a portion of the IV line, such as the needle and/or a hub/connector/fitting, as the body 102 is placed in close proximity to the point of entry of the IV line's need into the skin. The body 102 then acts in somewhat of a wedge-like fashion, to be positioned between the IV line and the skin, to separate the IV line from the skin, to support the IV line above the skin, and to distribute pressure that would be exerted on the skin by the IV line over a relatively larger area, as will be appreciated from FIG. 5.

In the exemplary embodiment of FIG. 3, the body 102 does not have a uniform thickness, but rather is tapered to have a relatively lesser thickness near the window 104 and a relatively greater thickness further from the window 104. This facilitates use of the body in somewhat of a wedge-like fashion, to support the IV line/needle at an angle relative to the skin and to lift and support the IV line from/above the skin. In the example of FIG. 4, a first portion 120 of the body 102 is tapered toward the window 104 according to a first angle A, and a second portion 130 of the body is tapered according to a second angle B that is greater than the first angle A. In the example of FIG. 5, a first portion 120 of the body 102 is tapered toward the window 104 according to a first angle A, and a second portion 130 of the body is tapered according to a second angle B that is less than the first angle A.

The embodiments of FIGS. 3 and 4 may be used as shown in FIG. 5. In the example of FIG. 5, the IV tape 30 is placed over the catheter 12, a portion of the hub/connector 14, and a portion of the protection device 100, to fix the IV line 10 and skin and nerve protective device 100 to the skin.

In any of the embodiments shown, the lower surface 106 may be provided/coated with a field of adhesive for adhering/fixing the protective device 100 to the skin. The adhesive may be a pressure sensitive adhesive, and the field of adhesive may be provided with a removable release sheet that initially covers a surface of the adhesive field and is removable to expose the adhesive for forming an adhesive bond with the skin at an appropriate time. In certain embodiments, this may be sufficient to eliminate a need for use of the IV tape 30, although it may be desirable to retain it to use the tape to prevent against infection at the penetration site.

Further, in any of the embodiments shown, the upper surface 108 may be provided/coated with a field of adhesive for adhering/fixing the IV line (or its component parts) to the IV nerve protective device 100. The adhesive may be a pressure sensitive adhesive, and the field of adhesive may be provided with a removable release sheet that initially covers a surface of the adhesive field and is removable to expose the adhesive for forming an adhesive bond with the skin at an appropriate time. In certain embodiments, this may be sufficient to eliminate a need for use of the IV tape 30.

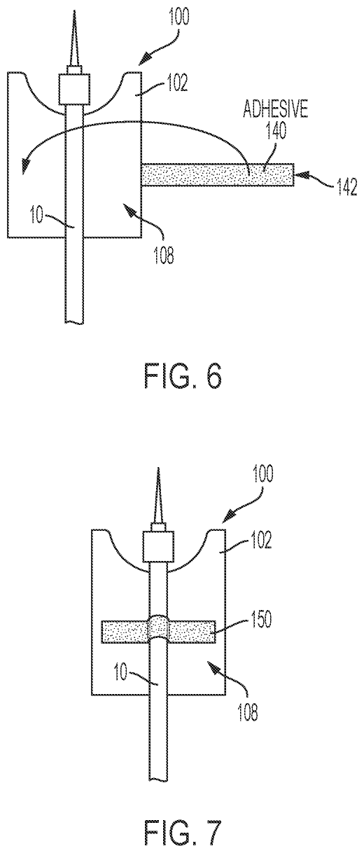

FIGS. 6 and 7 are top views of other exemplary IV skin and nerve protective devices in accordance with alternative embodiments of the present invention. In the example of FIG. 6, the protective device 100 further includes a securing tab 140, which may be added to any embodiment discussed above. The securing tab 140 may be fixed to the body at one end, and there may be adhesive 142, on the upper surface 108 and/or on the securing tab 140 (e.g., with a release sheet), such that the securing tab 140 may be folded over to overlie an IV line 10, and then be secured to the upper surface 108 of the body 102, to secure the IV line 10 to the protective device 100.

In the example of FIG. 7, the protective device 100 further includes a catch 150 which may be added to any embodiment discussed above. The catch 150 may be a discrete body separate from the body 102, and there may be adhesive, on the upper surface 108 and/or on the catch 150 (e.g., with a release sheet), such that the catch may be placed to overlie an IV line 10, and then be secured to the upper surface 108 of the body 102, to secure the IV line 10 to the skin and nerve protective device 100.

Accordingly, in use, the protective device 100 may be positioned with its lower surface 106 abutting and extending along/overlying the patient's arm (or other body part, as needed) (see e.g., FIG. 1) adjacent a penetration point (or intended penetration point) of an IV line's needle 12. After (or before in the case of the embodiment of FIGS. 1 and 2) the needle has been placed to penetrate the patient's skin, the protective device 100 may be positioned between the IV line 10 and the patient's skin, preferably with the hub/connector 14 overlying the top surface 108 of the body 102 of the protective device 100 and with a portion of the IV line passing through the window 104. This may be performed after removing any release sheet from the lower surface 106 to expose any adhesive for adhering the IV skin and nerve protective device 100 to the skin.

IV tape may then be placed to fix the IV line and the protective device 100 to the skin, in certain embodiments. Alternatively, the IV line maybe fixed by pressing the IV line against the top surface 108 of the IV skin and nerve protective device (in embodiments including adhesive on the upper surface 108), and/or adhering the securing tab 140 or catch 150 to the upper surface 108 of the IV nerve protective device (in certain embodiments), as appropriate.

Accordingly, it will be appreciated that the IV nerve protective device acts to separate the IV line from the patient's skin/tissue and provide cushioning and/or pressure spreading to protect the patient from nerve compression and injury resulting from pressure exerted by IV lines on the skin.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A protective device for protecting a patient from injury resulting from pressure exerted on bodily tissue by an IV line, said device comprising:
a body defining a window dimensioned to receive a first portion of an IV line, said body having a lower surface having an area greater than a second portion of said IV line, said second portion being spaced from the bodily tissue and supported on said lower surface by said body when said IV line is in use with bodily tissue with said first portion positioned within said window.

2. The device of claim 1, wherein said body is constructed of a compressible material.

3. The device of claim 1, wherein said body has a peripheral edge, and wherein said window is defined to be open to said peripheral edge.

4. The device of claim 1, wherein said body has a peripheral edge, and wherein said window is defined to be wholly internal to said peripheral edge.

5. The device of claim 1, further comprising adhesive disposed on said lower surface.

6. The device of claim 1, said body having an upper surface, said body further comprising adhesive disposed on a portion of said upper surface.

7. The device of claim 1, wherein a thickness of said body is not uniform.

8. The device of claim 1, wherein said body is tapered to have a relatively lesser thickness nearer said window and a relatively greater thickness further from said window.

9. The device of claim 1, said body having an upper surface, said device further comprising a securing tab having a first end secured to said body and a free end securable to said upper surface of said body.

10. The device of claim 1, said body having an upper surface, said device further comprising a catch securable to said upper surface of said body.

11. A protective device for protecting a patient from injury resulting from pressure exerted on bodily tissue by an IV line, said device comprising:
a body defining a window dimensioned to receive a first portion of an IV line, said body having a lower surface having an area greater than a second portion of said IV line, said body being configured to support said second portion on said bodily tissue when said IV line is in use with bodily tissue with said first portion positioned within said window.

12. The device of claim 11, wherein said body has a peripheral edge, and wherein said window is defined to be open to said peripheral edge.

13. The device of claim 11, wherein said body has a peripheral edge, and wherein said window is defined to be wholly internal to said peripheral edge.

14. The device of claim 11, further comprising adhesive disposed on said lower surface.

15. The device of claim 11, said body having an upper surface, said body further comprising adhesive disposed on a portion of said upper surface.

16. The device of claim 11, wherein a thickness of said body is not uniform.

17. The device of claim 11, wherein said body is tapered to have a relatively lesser thickness nearer said window and a relatively greater thickness further from said window.

18. The device of claim 11, said body having an upper surface, said device further comprising a securing tab having a first end secured to said body and a free end securable to said upper surface of said body.

19. The device of claim 11, said body having an upper surface, said device further comprising a catch securable to said upper surface of said body.

20. A protective device for protecting a patient from injury resulting from pressure exerted on bodily tissue by an IV line, said device comprising:
a body defining a window dimensioned to receive a first portion of an IV line, said body having a lower surface having adhesive disposed thereon, said lower surface having an area greater than a second portion of said IV line, said body being configured to support said second portion on said bodily tissue when said IV line is in use with bodily tissue with said first portion positioned within said window.

21. The device of claim 20, wherein said body has a peripheral edge, and wherein said window is defined to be open to said peripheral edge.

22. The device of claim 20, wherein a thickness of said body is not uniform.

23. The device of claim 20, said body having an upper surface, said device further comprising a securing tab having a first end secured to said body and a free end securable to said upper surface of said body.

24. The device of claim 20, said body having an upper surface, said device further comprising a catch securable to said upper surface of said body.
